(19) United States
(12) Patent Application Publication
(10) Pub. No.: US 2024/0407684 A1
(43) Pub. Date: Dec. 12, 2024

(54) ANALYTICAL DEVICES,SYSTEMS,MEDIA AND METHODS FOR MEASURING ANALYTES IN BIOLOGICAL FLUIDS

(71) Applicant: Roobik Azarnia, Niles, MI (US)

(72) Inventor: Roobik Azarnia, Niles, MI (US)

(21) Appl. No.: 18/206,259

(22) Filed: Jun. 6, 2023

(51) Int. Cl. A61B 5/157; A61B 5/1486; A61B 5/151; G01N 27/327

(52) U.S. Cl. CPC A61B 5/157; A61B 5/1486; A61B 5/151; G01N 27/3272

(57) ABSTRACT

A biochemical analysis instrument for fluid analyses such as whole blood, configured to receive a disposable test-lancet to firstly produce a small sample of biological fluid and subsequently to facilitate testing for the amount of an analyte in the fluid sample by sensing the amount of the analyte, wherein said test-lancet includes a sensor for sensing a reaction disposed in the test-lancet housing. Also a sample port is configured in the integrated test-lancet housing, the sample port being formed to provide access to the sensor area by capillary action. Said test-lancet housing is configured to be received by a lancing device integrated with the analysis instrument and is capable of moving the test-lancet forward to puncture a donors' skin, and is capable of directing the electrical signals generated by the sensor from the active zone of the test-lancet to the analysis instrument for evaluation and displaying said measurable change.

### CROSS-REFERENCE TO RELATED APPLICATIONS

This application claims the benefit of U.S. Provisional Application No. 63/349,952 filed Jun. 7, 2022.

### BRIEF DESCRIPTION OF THE DRAWINGS

With reference to the drawings, FIGS. 1-4 depict preferred embodiments of the improved, integrated test-lancet 5 of the instant invention. The test-lancet 5 represents an improvement over prior lancets. It embodies novel features that make the test-lancet 5 more integrated, easier to use, and less costly to manufacture. In a preferred use, a whole blood sample from a finger stick, or otherwise, is applied to the test-lancet to test for the presence, absence, and/or amount of a suspected analyte, e.g., glucose. It is important to note that whole blood may be tested for other analytes such as cholesterol or alcohol.

FIG. 1 is a conceptual illustration of an analyte measurement system or apparatus in accordance with a representative embodiment of present disclosure, which includes a disposable test-lancet 5 configured for mating engagement with and disengagement from a lancing device 3 and an analyte measurement device 1.

FIG. 2 is a schematic illustration of a representative test-lancet 5 in conjunction with a lancing device 3 and an analyte measurement device 1 according to an embodiment of the present disclosure.

FIG. 3 is a perspective view of a test-lancet 5 having a lancet 8 according to one embodiment of the present invention.

FIG. 4A is a cross-sectional view of test-lancet 5 illustrated in FIG. 3 designated as X-1.

FIG. 4B is a cross-sectional view of test-lancet 5 illustrated in FIG. 3 designated as X-2. In this figure the sample transfer port 9 is visible at the top and the venting pore 6 is visible at the side.

FIG. 4C is a cross-sectional view of test-lancet 5 illustrated in FIG. 3 designated as X-3.

### OVERVIEW

Embodiments in accordance with the present disclosure are directed to manual, semi-automated, or automated systems, apparatuses, instruments, structures, devices, and media for (a) receiving a small volume of a liquid as a fluid sample, which is typically a biological fluid such as whole blood or plasma; and (b) detecting the presence or estimating the level or quantity of one or more analytes in the received fluid sample.

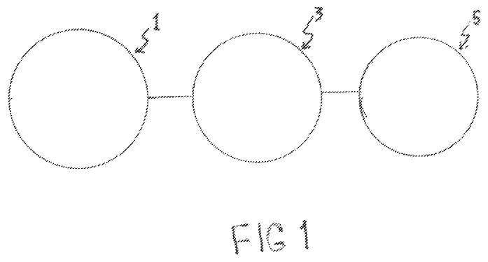

FIG. 1 is a conceptual illustration of an analyte detection, measurement, metering, or monitoring system or apparatus in accordance with a representative embodiment of present disclosure, which includes a disposable or replaceable integrated test-lancet that can be removably or matingly engaged with a portion of a body of an analyte meter or measurement instrument or device.

Depending upon embodiment details, the analyte measurement device can include a visual display device such as an LCD screen carried by its body, or exclude a visual display device. Internally, the analyte measurement device typically includes processing resources (e.g., a processing unit), data storage resources (e.g., a memory, in which program instructions that are executable by the processing unit can be stored), as well as power resources (e.g., a battery) and signal transfer/communication resources, in a manner readily understood by one of ordinary skill in the relevant art. In certain embodiments (e.g., in which a visual display device is not carried by the analyte measurement device's body, the analyte measurement device is configured for communicating analyte measurement results to a separate or remote display/processing system, device, or station having processing, memory, and communication resources, for instance, a desktop, laptop, or tablet computer, or a smart phone.

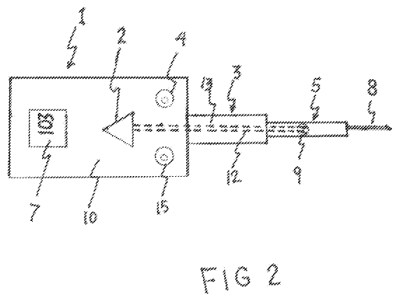

In a representative embodiment, the analyte measurement apparatus is configured for detecting, estimating, or measuring blood glucose levels (e.g., the analyte measurement device includes or is a blood glucose monitor). In other embodiments, the analyte measurement apparatus and/or the integrated test-lancet can be additionally or alternatively configured for detecting, estimating, or measuring the levels of one or more other analytes, such as blood cholesterol, alcohol or lactate. As further described in FIG. 2 and in more detail below, the test-lancet includes a housing that carries a test zone.

Each test zone includes or is a generally thin, fluid transport material or medium that can transport, distribute, or spread a fluid sample to which the test zone is exposed into, within, across, and/or at least substantially throughout the test zone. Such fluid transport can be facilitated or occur by way of absorption, wicking, capillary action, surface or surface tension effects, or adsorptive and/or another type of fluid transport mechanism (e.g., diffusion).

The fluid sample is typically volumetrically small or very small, for instance, less than or equal to 1-2 micro liters. Any given test zone can be loaded (e.g., pre-loaded) with one or more reagents or reagent chemical composition, including but not limited to enzymes, proteins, salts, surfactant(s), chemical stabilizers and other ingredients, in a manner readily understood by one of ordinary skill in the relevant art.

### BACKGROUND

The present disclosure generally relates to novel devices for estimating or measuring the level of an analyte within a fluid. A particular aspect of the present disclosure is directed to an analyte measurement instrument into which a removable/replaceable/disposable integrated test-lancet can be inserted. The test-lancet includes a test zone wherein includes a fluid transport material configured for receiving a small volume fluid sample, and rapidly transporting or distributing the fluid sample substantially throughout the test zone within the test-lancet housing. The integrated test-lancet additionally carries a set of sensors configured for measuring an analyte level within a test zone under consideration. The invention also provides an analysis device capable of receiving, dispensing and analyzing input from the test-lancet.

Detection and measurement of an appropriate analyte in a small sample of blood or other biological fluid samples, at home or near the patient has become commonplace in clinical medicine and is aiding in the diagnosing, monitoring and treatment of many diseases. One prominent example of this practice is the way diabetic patients are able to self-monitor and control their blood glucose to near normal levels at home or at work. This type of self-monitoring and glycemic control, significantly reduces the risk of developing serious complications related to diabetes as shown by landmark outcome studies.

The currently available blood glucose test-strips, primarily designed for electrochemical detection of glucose in conjunction with hand-held meters/devices, are capable of accurately sampling of a small volume of liquid in a precise manner for given batch of test-strips. The test-strips are designed for one time use only and each test-strip has built-in electrodes for a single use only after which the test-strip is discarded entirely.

Good blood glucose control, among other steps to be taken by the patient, requires routine self-monitoring of blood glucose levels at several daily intervals. Beginning in the early 1970's, relatively easy-to-use self monitoring devices provided blood glucose measurements by diabetics, allowing better control of blood glucose levels by utilizing the test results to determine proper insulin dosage or other medications to be administered. Early models of such blood glucose monitoring (BGM) devices were based on photometric principles of glucose detection, whereas more recent devices are biosensor (e.g. electrochemical) based in their operation.

In both versions of the BGM devices, generally a disposable, onetime-use reagent test-strip is used in conjunction with the analysis device. Such test-strips are generally small, flat and rectangular with the two ends serving separate functions. All such strips have a front end to receive a small volume of capillary blood from a finger stick. In addition, the front end contains an appropriate reagent mixture that reacts chemically with the analyte present in the blood sample, in this case glucose, to generate an appropriate signal; either a color change or an electrochemical signal proportional to the amount of analyte present in the fluid sample.

In the optical BGM devices, the end result of the chemical reaction is a color change detected and measured, whereas in the biosensor based BGM devices there is an electrochemical reaction taking place resulting in an electrical signal detected and measured. The other end of the test-strip is to be inserted into the BGM device to position the test-strip in the appropriate place within the device for optical measurement or in the case of biosensor based devices, to make electrical contact for the electrodes built into the strips with the electrical circuitry built into the analysis device to receive electrical signals.

There are a number of inherent disadvantages to the use of existing test-strips and associated devices:
1. The user must always carry an adequate number of test trips in a separate container from the analysis device, a lancing device and separately adequate number of lancets in yet a third container. That arrangement places additional burden on the end user and potentially with increased cost for each test.
4. The manufacturing process for most test-strips is elaborate and costly. In the case of the biosensor-based test-strips, the process requires additional steps and components for incorporating electrodes into the test-strips, hence increasing overall cost.
5. In addition to the requirement of a test-strip for each measurement the end user also must utilize separately a lancet for impaling and piercing a small area of the skin, typically of a finger tip, in order to generate a small volume of fluid, in the case of a diabetic a small volume of capillary blood in the range of 10-25 uL. This is a primary requirement in the protocol of all existing BGM's and points to the fact that the current BGM's require two separate steps and two separate one-time-use disposable parts, namely a piercing lancet to generate a small volume of blood and followed by a test-strip to receive a small fraction of the blood sample for analysis. As noted, the use of a integrated test-lancet eliminates the need for two separate parts and steps for a successful analysis by combining the sample retrieval and subsequent analysis of said sample into one integrated structure; namely an integrated test-lancet capable of generating and receiving said fluid sample and subsequently sensing and measuring the quantity of the analyte present in said fluid sample.
6. Careful examination of the cost structure of the current test-strips indicates that typically there are at a minimum 6-7 costly components both in terms of material cost and equipment and labor required for a complete test-strip. Reducing and/or eliminating this cost nearly entirely lowers the manufacturing cost of the integrated-test-lancet.

It would therefore be desirable to develop a method and apparatus for obtaining blood for diagnostic purposes in a less costly, more convenient and reliable manner.

Conventional lancing devices would have several disadvantages for an apparatus that combines the processes of lancing, fluid collecting, and analyte sensing into one automated instrument.

Conventional lancing devices, such as those described in U.S. Pat. Nos. Re. 32,922, 4,203,446, 4,990,154, and 5,487,748, accept commercially available, disposable lancets. Most conventional lancing devices are not integrated with a diagnostic instrument

### BRIEF SUMMARY OF THE INVENTION

The present invention includes a fluid sensing device and apparatus capable of receiving, dispensing and analyzing an integrated lancing and sensing device—a test-lancet, wherein the device includes an exposed test zone open to the ambient space capable of receiving a small sample of fluid. The integrated test-lancet is selectively exposed at the test zone, and whereby the test device is in contact with or in close proximity of an electrochemical sensor.

According to an aspect of the present invention there is provided a test device suitable for testing of analyte concentration in a fluid to be applied thereto, the test device includes a cavity having an active zone to which the fluid sample is to be applied by the sample donor. The active zone contains reagents which are specific and reactive to the desired analyte to produce an electrochemical signal proportional to the amount of the analyte present in the fluid sample.

Further, the object of this invention is to aid in obtaining an integrated analytical instrument with a built-in lancet-driver, capable of receiving a test-lancet for electrochemical analysis of fluids, capable of obtaining a relatively small fixed volume of liquid, e.g. biological fluids, including but not limited to whole blood or plasma, such that the fluid sample may interact chemically with the on-board electrodes pre-coated with biochemical reagent. The integrated test-lancet with on-board sensor will generate an electrochemical signal and will be discarded after each use.

FIG. 2 depicts a schematic illustration of one example of such a device. It is comprised of an analytical device connected to a lancet with built-in sensor capable of receiving a small volume of fluid sample material and generating an electrochemical signal detected by the device capable of measuring the signal from the sensor onboard the lancet and converting the signal to a meaningful digital output. The sensor module includes sensing elements themselves (e.g., electrodes), associated sensing signal transfer elements (e.g., electrical wiring), possibly sensing signal conditioning elements (e.g., signal filters), and/or other elements. The sensor module can be configured to interface with a signal communication interface carried by the lancet driver and/or the lancet, such as a multi-pin (e.g., 4 pin) electrical interface that can mate with a corresponding interface of the analyte measurement device.

In a number of embodiments, the test-lancet and/or the analyte measurement device include a set of mating engagement, locking, latching, or retaining elements or structures that facilitate simple, rapid, and secure engagement of the test-lancet with a lancing device present at one end of the analyte measurement device, as well as simple, rapid disengagement of the test-lancet there from. The mating engagement elements can facilitate correct insertion of test-lancet into the lancing device, such that consistent or reliable analyte measurements can be made after each test-lancet replacement event.

### DETAILED DESCRIPTION OF THE INTEGRATED TEST-LANCET SYSTEM

Examples of embodiments in accordance with the present disclosure described herein are non-limiting representative examples that are provided for purpose of aiding clarity and understanding. Additional or other embodiments or embodiment details not expressly described herein that conform to the fundamental structural and/or functional principles of embodiments described herein fall within the scope of the present disclosure.

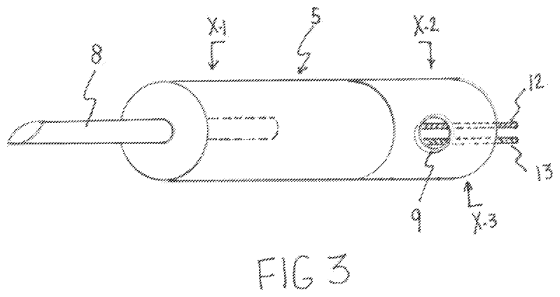

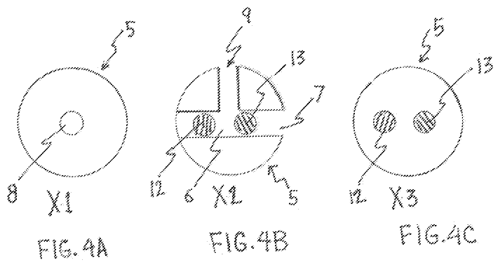

Referring now more specifically to FIG. 2, which shows a blood glucose test-lancet dispensing and analysis device 1 embodying the present invention. The test-lancet dispensing and analysis instrument 1 is a handheld device which includes a housing 10 with ON/OFF 4 and memory/control buttons 15 as known in the art. A test-lancet housing area 5 is formed in housing 10. A hinged lancing device 3 is mounted on the lower side of the housing 10 capable of selectively connecting with the housing 10 at its distal end and also selectively connecting and receiving a disposable test-lancet 5 at its proximal end. Housing area 5 is sized, oriented and shaped to receive and operatively retain a test-lancet 5 therein in operative relationship to a test head (not shown) mounted within lancet-driver housing 3. In this manner the trio components—the test-lancet 5, the lancet driver 3 and the analytical device 1, connect physically and operationally, and the interiors are electrically connected via the conductive pathways 12 and 13, such that functionally the active zone 6 present in the test-lancet 5 (FIG. 4A) is connected with the electronic circuitry analysis component 2 (FIG. 2) within instrument 10 to receive electrical pulses/currents generated at the active zone 6 are detected and analyzed. A liquid crystal display window 2 (FIG. 2) is disposed on housing 10 to display messages and analysis test results. The test-lancet 5 is movable allowing the thin needle 8 (FIG. 3) to travel outward to puncture a donor's skin in order to produce a small drop 5-25 uL of the user's blood. The test-lancet 5 and the needle 8 is then retracted to its prior location and a drop of blood is delivered by the sample donor to its sample delivery port 9 (FIG. 3 and FIG. 4B) opening and is moved by capillary or wicking action into the test-lancet active zone 6 area configured within the interior of the test-lancet 5. Rapid mixing and biochemical reaction ensues thereafter within the active zone 6 (FIG. 4B) generating an electrochemical signal travelling via electrodes 12 and 13 to the analyzer 1 circuitry 2 (FIG. 2) for analysis and signal proportional to the concentration of the analyte is displayed at the LCD 7.

Additional details presented below:
A test-lancet housing 5 that generally can be manufactured utilizing a mold injection method from a plurality of polymeric/plastic material.
A lancing needle 8 presently extending outward from the test-lancet housing 5 can be readily manufactured from a variety of metallic material such as stainless steel and similar material, in a manner readily understood by one of ordinary skill in the relevant art.
Metallic and/or other conductive material 12 and 13 present at the active zone 6 (FIG. 4B) can extend through the entire length of said housing 5 and are exposed to the exterior space at the sample port 9 and are covered and can be factory pre-coated with the reagent 11 present at the active/test zone 6 (FIG. 2 and FIG. 4B—not shown).
A sampling port 9 (FIG. 2 and FIG. 4B) is configured for receiving a small, 1-2 uL sample of fluid for analysis when presented at the sample port 9 by the donor and flowing rapidly leading to the sensor configured at test zone 6, instantly mixing with the reagent mixture 11 therein producing an electrochemical signal proportional to the concentration of the analyte present in the fluid sample.
A minimum of two electrodes built into the body of the test-lancet 5 whereby the working and reference electrodes 12 and 13 are in physical/chemical contact with a test fluid at the sensor/test zone 6 (FIG. 4B and FIG. 4C), and in conductive contact with the circuitry 2 configured in test meter/device 10, at the other end of the electrodes.
A pair of electrodes working 12 and reference 13 can be made of any conductive material, such as metal, carbon or similar material in a manner readily understood by one of ordinary skill in the relevant art, are incorporated throughout the distance from test zone 6 through the lancing device 3 reaching the electrical contact points at the circuitry 2 configured in the analysis device 10.
A test-lancet 5 housing that may contain desiccant material within the active zone 6.
A test-lancet housing 5 that is equipped with an access port 9 for receiving fluid samples from a donor.
A piercing needle 8 made of solid metallic material is located at the front end of the test-lancet housing 5 capable of piercing the outer layer of a donor skin such as a finger tip, in order to produce a small sample of fluid at the site of impalement.
A test-lancet housing 5 capable of anchoring/coupling physically with the proximal end of a lancing device 3 and establishing a conductive pathway by engaging and making contact with the electrodes present in the body of the lancing device 3, and said lancing device 3 is capable of receiving a test-lancet 5 and capable of spring loading said test-lancet 5 and subsequently releasing said test-lancet 5 against an area of a donor's skin in order to generate a small volume of fluid sample.
A lancing device 3 that is physically in contact with the front end of an analytical device/meter housing 10 and is capable of interlocking with the analytical device 10 at its distal end and a test-lancet 105 at its proximal end.
Analytical device 10 having a lancing device 3 connected at its front end and capable of receiving and electrical signals/currents generated at the test zone 6 of the test-lancet 5, capable of transmission through the lancing device 3.
A device 1 with CPU built-in and other parts/components capable of receiving such electronic inputs/signals and analyzing said inputs and measuring the concentration of a given analyte present in the fluid sample presented at the test zone 6.

1. A test-lancet analytical device capable of creating a sample of donor blood and subsequently detecting and measuring analyte of choice in said blood sample, said test-lancet device comprising:
a housing, said housing having a sensor/sensing elements configured therein for receiving a sample of the donor fluid for biochemical analysis; and
wherein said sensor is comprised of a conductive electrodes comprising a working electrode and a reference electrode, wherein the working electrode is configured to generate an electrochemical signal output indicative of the analyte concentration;
a needle protruding at the proximal end of said housing; and
a lancing device having mechanical and electrical means of coupling with the distal end of said housing; and
an analytical device configured at the distal end of said lancing device.

2. The test-lancet of claim 1, wherein the test-lancet housing has a proximal end and a distal end, said proximal end having a protruding sharp needle capable of puncturing a donor's skin, and said distal end having mechanical and physical means of coupling and contacting a lancet driver/lancing device housing.

3. The lancet driver housing of claim 2, capable of spring loading said test-lancet and subsequently releasing said test-lancet against an area of a donor's skin in order to generate a small volume of fluid sample.

4. The lancet driver housing of claim 2, at its distal end located at the juncture with an analysis device/instrument is configured for anchoring/coupling with the proximal end of said analysis device and establishing a conductive pathway for electrical signals generated from test-lancet active zone to said analysis device.

5. The test-lancet of claim 1, wherein two or more electrodes are disposed in said housing, at least two electrodes including a working electrode, and a counter electrode.

6. The electrode configuration of claim 4, in yet another embodiment is configured to four electrodes including a fill-detect anode, and a fill-detect cathode.

7. The test-lancet of claim 1, wherein a plurality of electrical contacts disposed on said housing, said plurality of electrical contacts including a working electrode contact, a counter/reference electrode contact, a fill-detect anode contact, and a fill-detect cathode contact;

8. The test-lancet of claim 1, wherein a plurality of conductive traces/means are disposed in said housing, said plurality of conductive traces electrically connecting said working electrode to said working electrode contact configured on the proximal end of the lancing device, said counter electrode to said counter electrode contact, said fill-detect anode to said fill-detect anode contact, said fill-detect cathode to said fill-detect cathode contact therein.

9. The test-lancet housing of claim 1, having a pit so as to define an exposed working electrode portion and an exposed counter electrode portion within; said working electrode, said counter electrode, said fill-detect anode, and said fill-detect cathode being disposed in said pit.

10. The pit of claim 7, wherein said reagent layer is disposed in said pit and covers said exposed working electrode portion and includes an enzyme, a mediator, surfactant(s), other components and said reagent causes a reaction which generates an electrochemical signal when presented with the fluid sample containing the analyte.

11. The pit of claim 7, wherein said pit defines a testing zone for testing a blood sample, said pit being dimensioned to draw said blood sample in through said port at proximal end of said pit by capillary or wicking action.

12. The test-lancet housing of claim 1, wherein said active/test zone is configured with a venting member, said venting member shaft being disposed between adjacent active zone and the exterior space.

13. The test-lancet of claim 1, wherein the dimensions of the active/test zone are adapted to accommodate a sample volume of about 1 μl or less.

14. The test-lancet of claim 1, wherein the housing is configured with a sample receiving port modified with the means for transporting sample liquid via a capillary channel/conduit or a wick of absorbent material for receiving the fluid sample from a donor to fill the entire volume of the test zone.

15. A piercing needle of claim 2, wherein said needle is made of solid metallic material and is capable of piercing the outer layer of a donor skin such as a finger tip, in order to produce a small sample of fluid at the site of impalement.

16. A piercing needle of claim 2, wherein said needle is made non-metallic material and is capable of piercing the outer layer of a donor skin such as a finger tip, in order to produce a small sample of fluid at the site of impalement.

17. A test-lancet housing capable of anchoring/coupling physically with the proximal end of a lancing device and establishing a conductive pathway by engaging and making contact with the electrodes present in the body of the lancing device, and said lancing device is capable of receiving a test-lancet and capable of spring loading said test-lancet and subsequently releasing said test-lancet against an area of a donor's skin in order to generate a small volume of fluid sample.

18. A lancing device that is physically in contact with the front end of an analytical device/meter housing and is capable of interlocking with the analytical device at its distal end and a test-lancet at its proximal end.
